(19) United States
(12) Patent Application Publication
(10) Pub. No.: US 2009/0312801 A1
(43) Pub. Date: Dec. 17, 2009

(54) MULTI-GUIDE PLATE HOLDER

(76) Inventors: Jeremy J. LEMOINE, Austin, TX (US); Keith R. Knippa, Austin, TX (US); Dennis C. Moad, Manor, TX (US); Larry O. Fisher, Smithville, TX (US)

Correspondence Address: SPRINKLE IP LAW GROUP, 1301 W. 25TH STREET, SUITE 408, AUSTIN, TX, 78705, US

(21) Appl. No.: 12/139,607

(22) Filed: Jun. 16, 2008

(51) Int. Cl. A61B 17/80; A61B 17/04; A61B 17/32

(52) U.S. Cl. 606/280; 606/301; 606/167

(57) ABSTRACT

Methods, instruments, and kits for attaching cranial plates. One embodiment implements a method which includes releasably attaching a plate to an instrument (using retaining members of the instrument) and placing the plate on a cranium (using an elongated and offset handle of the instrument). The method can include drilling a hole in the cranium, tapping the hole, and placing the screw in the hole using the instrument which defines a pattern of holes corresponding to, a pattern of holes on the plate. The drilling of the hole, tapping of the hole, and placing of the screw can occur by engaging one of the instrument holes with, respectively, a drill bit shank, a tap shank, and a screw head having a common diameter corresponding to a diameter of the instrument hole. The method can include selecting the plate, the drill bit, the tap, and the screw from a kit.

### TECHNICAL FIELD

This disclosure relates generally to cranial surgery methods and instruments, and more particularly to surgical methods and instruments for attaching cranial plates to occipital bones.

### BACKGROUND

Trauma and degenerative conditions can sometimes indicate the desirability of fixing a portion of a patient's cervical spine against movement and, sometimes, securing portions of the cervical spine and the patient's cranium against movement relative to each other. In such situations, cranial plates can be attached to the occipital bone of patients' craniums to secure cervical spine stabilization system components to the patients' craniums. In addition, cranial plates may be indicated to replace bone flaps following craniotomies in which they were created. The desirability for using a cranial plate in these, and other, situations may be determined by surgical or medical personnel based on an evaluation of the patient's condition.

When patient conditions indicate the desirability of using a cranial plate, and after the patient is prepared for surgery, the surgical site may be opened. Attachment holes may be drilled in the patient's occipital bone into which screws can be driven to attach a cranial plate to the occipital bone. The cranial plate can then be attached to the occipital bone using various screws. Inaccuracies associated with the placement of the attachment holes relative to each other, the angles at which the attachment holes are drilled, the diameters of the attachment holes, and other factors associated with creation of the attachment holes can cause the installed screws to impart stresses to the occipital bone, thereby potentially causing patient discomfort. Such inaccuracies can also affect the strength, functioning, and mechanical integrity of the spinal stabilization systems attached to the cranial plates. For instance, inaccurate installation can limit the range of motion allowed by the spinal stabilization system.

To create the attachment holes, surgical personnel can select a suitable location on the occipital bone based upon patient conditions. Surgical personnel can use a drill to create each attachment hole at the selected location. Surgical personnel can tap each attachment hole to thread it for the screws. Surgical personnel can then place the cranial plate over the pattern of attachment holes in the cranium and attempt to align the screw holes on the cranial plate with the attachment holes in the occipital bone. Using a screwdriver or other suitable device, surgical personnel can drive screws through the holes on the cranial plate and into the attachment holes created in the occipital bone. As noted above, inaccuracies generated in locating and creating the attachment holes can cause misalignments' between the plate holes and the attachment holes, thereby affecting the strength, functioning, and mechanical integrity of any spinal stabilization system which might be attached to the cranial plate.

Moreover, in the relatively crowded space adjacent to the patient's occipital bone, several instruments, surgical personnel, etc. must cooperate to attach the cranial plate to the occipital bone. For instance, one or more instruments may be necessary to locate desired locations for the attachment holes. Other instruments and surgical personnel can be involved in threading the attachment holes. Still other instruments and surgical personnel can be involved in locating the cranial plate accurately in relation to the attachment holes and holding the cranial plate in place for subsequent steps. When surgical personnel desire to place the screws, other instruments and surgical personnel can be involved in driving the screws through the plate hole and into the attachment holes. As a result of the number of instruments and surgical personnel potentially involved in attaching the cranial plate to the occipital bone, the surgical site can become crowded with instruments, hands, and surgical devices during the attachment of the cranial plate to the occipital bone. The number of instruments, surgical personnel, and surgical devices can complicate and slow the surgical procedures involved in attaching the cranial plate to the occipital bone.

### SUMMARY

Embodiments of the present disclosure provide methods, instruments, and kits of instruments for cranial surgery that eliminate, or at least substantially reduce, the shortcomings of previously available methods, instruments, and kits of instruments for cranial surgery.

Various embodiments provide instruments for use with cranial plates to improve the accuracy, precision, and efficiency of attaching cranial plates to occipital bones. In some embodiments, the instrument provides holes corresponding to holes in a cranial plate(s) and which have a diameter corresponding to a common diameter of a drill bit, a tap, and a screw used, respectively, to drill an attachment hole in the cranium, to tap the attachment hole, and to attach the cranial plate to the cranium. In some embodiments, the instrument includes an adapter which defines the instrument holes and an elongated handle which is offset from the adapter. The adapter can also include retaining members with which the cranial plate can be releasably attached to the instrument. The retaining members can be resilient fingers or set screws. The adapter can include a key corresponding to a key on the cranial plate to assist with aligning the plate holes with the attachment holes.

In methods implemented by embodiments, surgical personnel can releasably attach the cranial plate to the distal end of the adapter, use the instrument to place the cranial plate on the cranium at a selected position, and attach the cranial plate to the cranium. In attaching the cranial plate, surgical personnel can hold the instrument and plate in a selected instrument hole while engaging the instrument hole with the drill bit. As the drill bit engages the instrument hole, the instrument hole aligns the drill bit with the corresponding plate hole thereby accurately and precisely aligning the drill bit with the desired location of the attachment hole to be created. Surgical personnel can then drill the attachment hole to a desired depth, remove the drill bit, and begin the tapping the attachment hole. Surgical personnel can engage the instrument hole with the tap shank and, because of the common diameter, accurately and precisely align the tap with the attachment hole. Surgical personnel can tap the attachment hole, remove the tap, and begin placing a screw in the attachment hole. Surgical personnel can place a screw (tip first) in the instrument hole and push it toward the cranium with a screwdriver. The screw head can engage the instrument hole and, because of the common diameter, can be aligned accurately and precisely with the attachment hole. Surgical personnel can drive the screw into place using a screwdriver and repeat the process at each instrument hole until all attachment holes have been drilled and screws driven into each one. Because the cranial plate and instrument can remain attached to each other during various steps of the attachment process, each attachment hole can be accurately and precisely aligned with the plate and each other.

Various embodiments provide surgical kits for attaching cranial plates to occipital bones. Kits can include sets if cranial plates, screws, drill bits, taps, and various instruments. Each instrument can have an adapter with instrument holes through the body of the adapter. The screws heads, drill bit shanks, and tap bodies can have a common diameter corresponding to the diameter of the instrument holes. When the screws, drill bits, and taps differ in overall or nominal size, they can still have a common diameter.

Embodiments provide advantages over previously available approaches to attaching cranial plates. Cranial plates can be attached accurately and precisely by instruments of embodiments. Surgery can be efficient and quick while errors caused by misaligned drill bits, taps, screws, etc. can be eliminated or, at least, reduced by embodiments. Relative to each other, screws can be set more accurately and precisely by embodiments. Crowding of the surgical site with various instruments, drills, taps, portions of adjacent patient anatomy (such as the patient's shoulders), etc. can be reduced by embodiments.

### BRIEF DESCRIPTION OF THE DRAWINGS

A more complete understanding of the present disclosure and the advantages thereof may be acquired by referring to the following description, taken in conjunction with the accompanying drawings in which like reference numbers indicate like features and wherein:

FIG. 1 depicts a craniumi of a patient.

FIG. 2 depicts a cranial plate of some embodiments.

FIG. 3 depicts an instrument and drill of some embodiments for attaching cranial plates to craniums.

FIG. 4 depicts a cranial plate and an instrument of some embodiments for attaching cranial plates to craniums.

FIG. 5 depicts an instrument, a drill, a tap, and a screw of some embodiments for attaching cranial plates to craniums.

FIG. 6 depicts an instrument of some embodiments for attaching cranial plates to craniums.

FIG. 7 depicts an instrument of some embodiments for attaching cranial plates to craniums.

FIG. 8 depicts an instrument of some embodiments for attaching cranial plates to craniums.

FIG. 9 depicts a method for attaching a cranial plate to a cranium of some embodiments.

FIG. 10 depicts an instrument of some embodiments for attaching cranial plates to craniums.

FIG. 11 depicts an instrument of some embodiments for attaching cranial plates to craniums.

### DETAILED DESCRIPTION

The disclosure and the various features and advantageous details thereof are explained more fully with reference to the non-limiting embodiments detailed in the following description. Descriptions of well known starting materials, manufacturing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

As used herein, the terms “comprises,” “comprising,” “includes,” “including,” “has,” “having” or any other variation thereof, are intended to cover a non-exclusive inclusion. For example, a process, process, article, or apparatus that comprises a list of elements is not necessarily limited only those elements but may include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, “in one embodiment”.

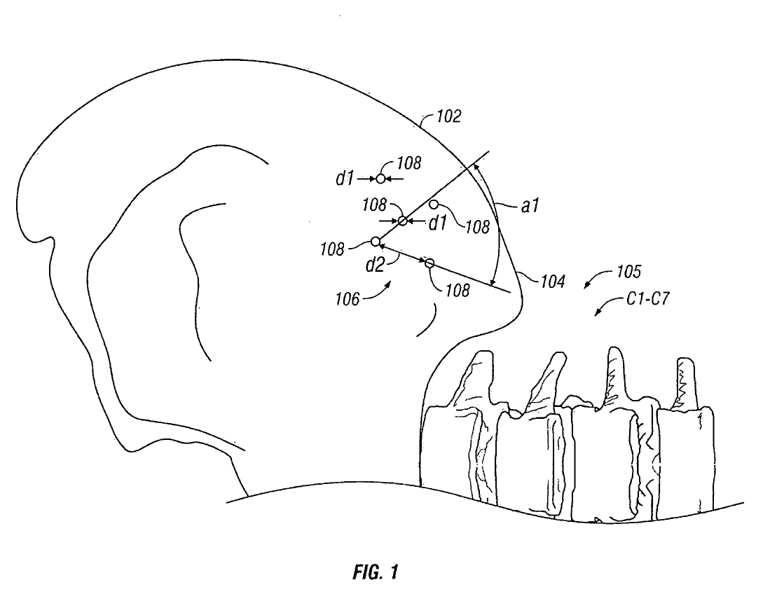

With reference now to FIG. 1, FIG. 1 depicts a patient's cranium 102 and more particularly occipital bone 104. Occipital bone 104 forms a posterior portion of cranium 102. Occipital bone 104 joins with cervical spine 105 at the atlas (cervical vertebra C1) and together with various other vertebrae (e.g., the axis or vertebra C2, and vertebrae C3-C7) of cervical spine 105 allows the patierit to flex, extend, and rotate the patient's neck and head. More particularly, atlas C1 and axis C2 allow the patient to rotate their head while other vertebra C3-C7 allow the patient to flex and extend their neck. Occasionally it may be necessary to partially, or entirely fix occipital bone 104 and one, or more, vertebrae C1-C7 of cervical spine 105 relative, to each other. For example, degeneration of one or more intervertebral discs, spondylolisthesis, stenosis, atlanto/axial fractures, incomplete or failed attempts to fuse such structures 104 and C1-C7 together, etc. may indicate some desirability of fixing occipital bone 104 to some vertebra C1-C7 of cervical spine 105, vertebrae of the thoracic spine, etc. In such scenarios, surgical personnel may recommend attachment of one or more rods to occipital bone 104 and cervical spine 105 to fix these anatomical structures 104 and 105 relative to each other. Such rods may be deemed static stabilization rods when they allow no, or little, relative movement between occipital bone 104 and cervical spine 105. Such rods may be deemed dynamic rods when they allow selected degrees of relative movement (longitudinal, extension, flexion, rotation, etc.) between occipital bone 104 and cervical spine 105. Many such spinal stabilization systems are known and exemplary spinal stabilization systems are offered by Abbott Spine of Austin, TX.

Surgical personnel often recommend using a plate to attach stabilization rods to occipital bone 104 or to replace bone flaps following craniotomies. To do so, surgical personnel can recommend creating pattern 106 of attachment holes 108 in occipital bone 104 to match corresponding patterns on various plates. Hole pattern 106 can be any pattern deemed sufficient to attach a plate to occipital bone 104. In the embodiment illustrated by FIG. 1, hole pattern 106 includes a centrally located attachment hole 108 and 4 equally sized attachment holes 108 distributed evenly about the central attachment hole 108. Attachment holes 108 can be tapped to accept screws which can attach the plate to occipital bone 104. Surgical personnel may desire that each attachment hole 108 have a diameter d1 (as illustrated by FIG. 1) which corresponds accurately enough to the nominal diameter of such screws so that the screws can hold the plate securely to occipital bone 104. Diameters d1 of attachment holes 108 may differ from each other though without departing from the scope of embodiments disclosed herein. Hole pattern 106 may define various angles and distances between various attachment holes 106 such as angle a1 and distance d2 between two particular attachment holes 108.

Surgical personnel may desire that attachment holes 108 be located relative to each other and to corresponding holes in the plate sufficiently accurately so as to impart little, or no, stress on occipital bone 104 when the plate is secured to occipital bone 104 by attachment holes 108. Should more stress than is practicable be imparted to occipital bone 104 by the screws, the patient may experience discomfort, increased post operative recovery time, etc. In addition, misalignment between the plate, attachment holes 108, the screws, etc. can cause surgical personnel to desire to undo and repeat certain steps of the plate attachment procedure. However, because portions of the patient's anatomy may be involved in such steps, circumstances may preclude (or limit) the ability of surgical personnel to remediate such situations. As a result, in some situations, it may be more desirable to leave the plate attached as is rather than attempt to remediate the situation.

Prior to attaching plates to occipital bones 104, surgical personnel may evaluate occipital bones 104 using patient interviews, information regarding patient medical history, palpations and X-ray, CT, CAT, MRI, etc. imaging techniques. Surgical personnel can evaluate whether attachment of plates, stabilization rods, etc. might treat, cure, or improve the underlying condition. Surgical personnel may, when they judge that attaching plates to occipital bones 104 may be likely to be beneficial, make initial selections of certain plates, screws, and techniques to use in treating the underlying conditions. For instance, surgical personnel may make an initial choice of plate configuration and size and a selection of screw configuration and length based on various information gleaned from the examinations. When desired, surgical personnel may prepare the patient for surgery and place the patient on an appropriate operating surface. When surgical personnel desire to approach occipital bone 104 from a posterior direction, the patient may be placed on the operating surface in a face down position.

With continuing reference to FIG. 1, creating attachment holes 108 may involve the use of various instruments. Instruments may be used by surgical personnel to drill attachment holes 108, tap threads into attachment holes 108, and to align, insert, and drive screws into attachment holes 108. Each instrument may have to be aligned with, operated adjacent to, and removed from attachment holes 108 for each attachment hole 108. Thus, areas adjacent to occipital bone 104 may be crowded with various instruments, the hands and arms of surgical personnel, alignment devices, support structures, suction devices, etc. during creation of attachment holes 108 and attachment of a plate to occipital bone 104. Patient anatomy can aggravate the crowding of the area adjacent to occipital bone 104. For example, angles between cranium 102 and cervical spine 105 can limit the volume of space adjacent to occipital bone 104. Other anatomic structures of the patients such as the patient's shoulders (not shown) can also limit the volume of space available for surgical personnel to work in that is adjacent to occipital bone 104.

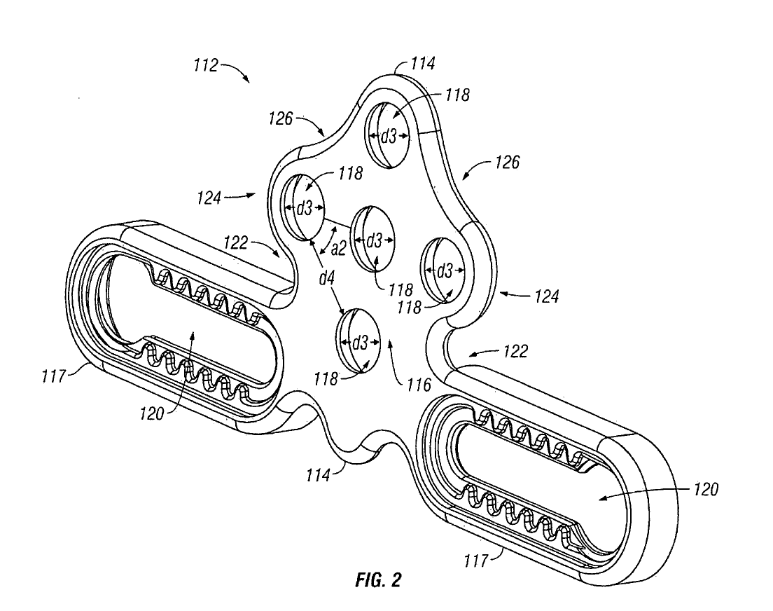

With reference now to FIG. 2, cranial plate 112 may be attached to occipital bone 104 in some embodiments. Cranial plate 112 may include a medially disposed body 114 shaped to correspond to selected areas of occipital bone 104. Body 114 may include hole pattern 116 of plate holes 118. Body 114 may define lobes around plate holes 118 which can spread loads which might otherwise be transferred to or from occipital bone 104 in more concentrated form. With regard to plate holes 118, diameters d3 of plate holes 118 may correspond to diameters d1 of attachment holes 108 in occipital bone 104 (see FIG. 1) so as to securely attach cranial plate 112 to occipital bone 104. Surgical personnel may desire that hole pattern 116 (on cranial plate 112) correspond accurately enough to hole pattern 106 (in occipital bone 104) so as to impart no, or minimal, stress (due to misalignments between plate holes 118 and attachment holes 108) to occipital bone 104. More particularly, surgical personnel may desire that angles a2 and distances d4 correspond sufficiently accurately to angles a1 and distances d2 respectively (of FIG. 1) so as to impart no, or minimal, stresses on occipital bone 104 when cranial plate 112 is attached to occipital bone 104. When desired, surgical personnel may navigate cranial plate 112 to a position adjacent occipital bone 104, align hole pattern 116 of cranial plate 112 with a desired location for hole pattern 106 of occipital bone 104, and place cranial plate 112 on occipital bone 104. As discussed herein, surgical personnel may create attachment holes 108 and attach cranial plate 112 to occipital bone with screws, bone anchors, bone hooks, etc. using attachment holes 108.

With continuing reference to FIG. 2, cranial plate 112 can include bosses 117 extending laterally from body 114. Bosses 117 can define apertures 120 through which attachment devices (for attaching stabilization rods to cranial plate 112) may extend and attach to cranial plate 112. Apertures 120 may include detents or other mechanisms to allow surgical personnel to adjust the position of various stabilization rods relative to cranial plate 112. In the embodiment illustrated by FIG. 2, bosses 117 include a number of detents allowing surgical personnel to adjust the spinal stabilization system to be attached to cranial plate 112 to accommodate patients. of varying sizes. More or less detents may be provided in bosses 117 without departing from the scope of embodiments disclosed herein. When installed in various spinal stabilization systems, cranial plate 112 may attach to occipital bone 104. Stabilization rods may attach to cranial plate 112 via bosses 120 and to selected vertebra C1-C7 of cervical spine 105 (see FIG. 1) via pedicle screws, bone anchors, bone hooks, etc.

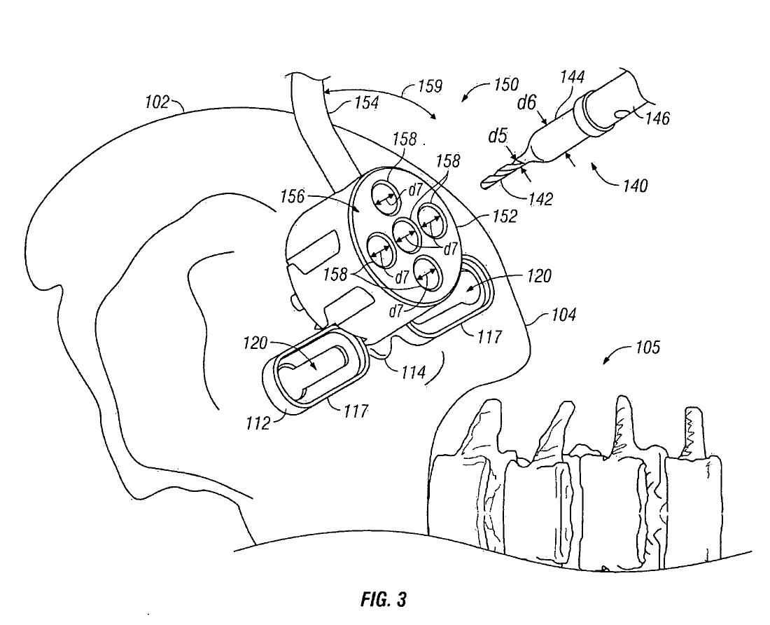

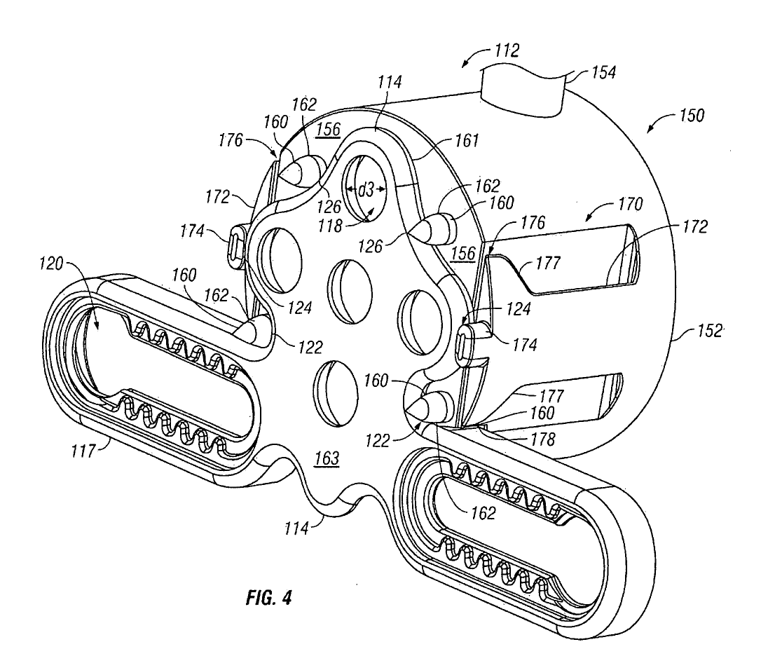

Cranial plate 112 can include features 122,124, and 126 which (as discussed with reference to FIG. 4) can aid in aligning cranial plate 112 with instruments for attaching cranial plate 112 to occipital bone 104. Features 124 can aid in attaching cranial plate 112 to instruments 150 discussed with reference to FIG. 3-9. Cranial plate 112 can be made of biocompatible materials such as titanium, stainless steel, zirconium, polymethyl methacrylate, etc.

With reference now to FIG. 3, FIG. 3 illustrates drill 140, instrument 150, and cranial plate 112 (releasably coupled to instrument 150) in relationship to cranium 102, occipital bone 104, and cervical spine 105 prior to attachment of cranial plate. 112 to occipital bone 104. Instrument 150 can be used to attach cranial plate 112 to occipital bone 104 while bosses 117 and apertures 120 may be used to attach stabilization rods to cranial plate 112. As discussed herein, drill 140 can be used in conjunction with instrument 150 to drill attachment holes 108 (see FIG. 1).

Drill 140 can include drill bit 142, drill bit shank 144, and extension 146 (through which a driving member may extend to drive drill bit 142). Drill bit 142 may have diameter d5 and drill bit shank 144 may have diameter d6 which can be greater than diameter d5. Still referring to FIG. 3, instrument 100 can include adapter 152 and extension 154. Adapter 152 of instrument 150 can define hole pattern 156 of instrument holes 158 which have diameter d7. Hole pattern 156 of instrument 150 may correspond to hole pattern 116 of cranial plate 112 and to hole pattern 106 in occipital bone 104. In the embodiment illustrated by FIG. 2, hole pattern 156 can include a centrally located plate hole 158 and 4 instrument holes 158 distributed equally about the central instrument hole 158 as shown. Instrument holes 158 can extend through adapter 152 to the surface of adapter 152 abutting cranial plate 112.

With continuing reference to FIG. 3, FIG. 3 illustrates directional arrow 159 according to which instrument 150 and cranial plate 112 can be rotated relative to each other to attach or detach cranial plate 112 from instrument 150. Attachment and detachment of cranial plate 112 to and from instrument 150 is discussed further with reference to FIG. 4. In some embodiments, cranial plate 112 remains attached to instrument 150 while attachment holes 108 are drilled, tapped, and used (in conjunction with certain screws) to attach cranial plate 112 to occipital bone 104. While instruments 150 are described herein as being suitable for attaching cranial plates 112 to occipital bones 104, it will be understood, that instruments similar to instrument 150 can be suitable for attaching other orthopedic plates to other anatomical structures such as other cranial bones (parietal bones, temporal bones, zygomatic bones, mastoid bones, etc.), and other bones whether healthy, injured or diseased as indicated by patient symptoms and patient evaluations by medical personnel. For instance, fractures may be treated by attaching plates to the affected bones using instruments and plates similar to those described herein without departing from the scope of the disclosure.

With reference now to FIG. 4, FIG. 4 illustrates cranial plate 112 attached to adapter 152 of instrument 150. Adapter 152 may have a distal face 156 which includes posts 160 projecting there from for aligning cranial plate 112 with adapter 152 and, more particularly, aligning instrument holes 158 with plate holes 118. When cranial plate 112 is attached to adapter 152, features 122 and 126 may abut alignment posts 160 to align cranial plate 112 with adapter 152 and, more particularly, to align plate holes 118 with instrument holes 158 (not shown). Alignment posts 160 may prevent movement of cranial plate 112 across distal face 156 of adapter 152. Alignment posts 160 can include arcuate portions 162 corresponding to the edges of cranial plate 114 to aid in aligning and retaining cranial plate 112 on adapter 152. As shown by FIG. 4, alignment posts 160 may extend beyond cranial plate 112 when cranial plate 112 is attached to adapter 152. Pointed tips of attachment posts 160 may grip pores, crevices, pits, depressions, etc. in the surface of occipital bone 104 so that plate 112 will tend to remain where it may be placed on occipital bone 104.

Alignment posts 160 and features 122 and 126 of cranial plate 112 may be keyed or otherwise configured to prevent attachment of cranial plate 112 to adapter 152 in relative positions other than user selected positions. For example, alignment posts 160 can be positioned on distal face 156 so that one pair abuts one lobe 161 of body 114 and so that another pair abuts another and differing portion of body 114 (such as neck 163 of body 114 adjacent to bosses 117). In the embodiment illustrated by FIG. 1, alignment posts 160 may allow attachment of cranial plate 112 in a position relative to adapter 152 in which bosses 117 lie on the side of cranial plate 112 which is opposite extension 154 of instrument 150.

With continuing reference to FIG. 4, FIG. 4 illustrates releasable attachment mechanism 170 of adapter 152. Attachment mechanism 170 can include resilient fingers 172 each including attachment posts 174. Resilient fingers 172 and the body of adapter 152 can define slots 176 therebetween. Slots 176 can be machined into adapter 152 or adapter can be cast with slots 176 pre-defined by appropriate features of the mold. Resilient fingers 172 and attachment posts 174 can be shaped and dimensioned so that attachment posts 174 abut features 124 of cranial plate 112 when cranial plate 112 is attached to adapter 152. Attachment posts 174 can include arcuate indentations (not shown) corresponding to features 124 of cranial plate 112 to aid in retaining cranial plate 112 on adapter 152. Resilient fingers 172 can be shaped and dimensioned to elastically yield when cranial plate 112 is pushed into the space between attachment posts 174 thereby allowing cranial plate 112 to be attached to adapter 152. Resilient fingers 172 can have a, user selected spring constant which can determine the force with which attachment fingers 172 press against features 124 of adapter 152. Such spring constants can be determined by the geometry, materials, etc. of resilient fingers 172.

To attach cranial plate 112 to instrument 150, cranial plate 112 can be aligned with alignment posts 160 (with features 122 and 126 generally adjacent to alignment posts 160) and attachment posts 174 (with features 124 adjacent to attachment posts 174). Cranial plate 112 can be pressed into place on adapter 152 between attachment posts 174. To detach cranial plate 112 from instrument 150, instrument 150 can be rotated relative to cranial plate 112 to pivot adapter 152 about the point(s) where it contacts bosses 117 of cranial plate 112, thereby lifting the proximal side of adapter 152 from cranial plate 112. Such rotation, as illustrated by directional arrow 159 (see FIG. 3) can cause features 124 of adapter 112 to overcome the grasping force of resilient fingers 172, thereby sliding passed attachment posts and releasing cranial plate 112 from instrument 150.

Thus, cranial plate 112 can be attached to instrument 150 by one user by that user holding instrument 150 in one hand and manipulating cranial plate 112 with the other, vice versa, or a combination thereof. One user may detach cranial plate 112 from instrument 150 by holding instrument 150 in one hand and manipulating cranial plate 112 with the other hand, vice versa, or a combination thereof. When cranial plate 112 is secured to a structure such as occipital bone 104, surgical personnel can detach instrument 150 from cranial plate 112 using one hand to manipulate instrument 150. In some embodiments, such surgical personnel can attach and detach cranial plate 112 and instrument 150 from each other without aid from other surgical personnel, thereby reducing the number of surgical personnel involved in at least some surgical procedures involved in attaching cranial plate 112 to occipital bone 104.

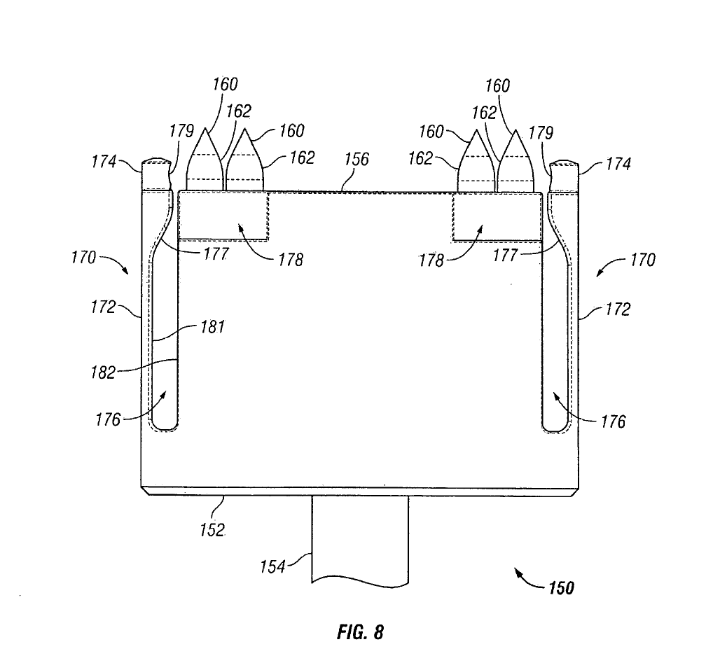

Resilient fingers 172 can include shoulders 177 which allow the edges of resilient fingers 172 to generally conform to the overall shape of distal surface 156 of adapter 152. Shoulder 177 can increase in width and depth as the distance on resilient finger 172 from distal face 156 decreases. Adapter 152 can define notch 178 (see FIG. 8) which can provide clearance for bosses 117 (with apertures 120) of cranial plate 112 when cranial plate 112 is attached to adapter 152.

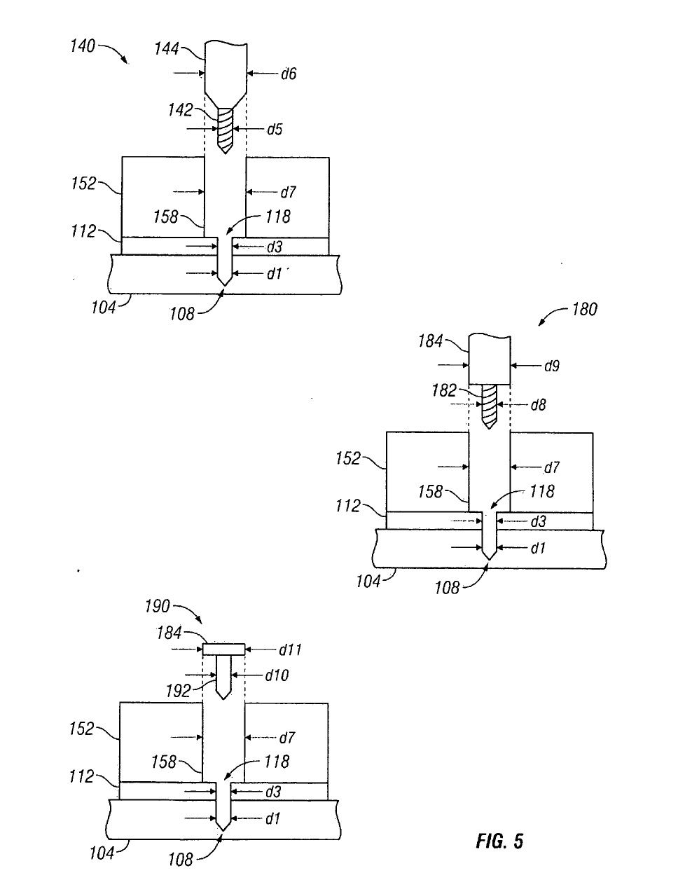

Now with reference to FIG. 5, FIG. 5 illustrates cross sectional views of instrument 150, cranial plate 112, and occipital bone 104 during various steps of attaching cranial plate 112 to occipital bone 104. FIG. 5 illustrates drill 140, tap 180, and screw 190 being used to attach cranial plate 112 to occipital bone 104. To attach cranial plate 112 to occipital bone 104, drill 140 can drill attachment hole 108, tap 160 can tap attachment hole 108, and screws 190 can attach cranial plate 112 to occipital bone 104 in conjunction with adapter 152 of instrument 150. Each attachment hole 108 can be drilled and tapped separately or in groups as surgical personnel may desire. Screws 190 can be driven into attachment holes 108 as each individual attachment hole 108 is created or in groups as groups of attachment holes 108 are created as may be desired. In one embodiment, attachment holes are, drilled one after the other and then tapped one after another.

In general, and as illustrated by FIG. 5, drill bit shank 144 diameter d6 can be greater than or equal to drill bit 142 diameter d5. Tap shank 184 diameter d9 can be greater than or equal to tap thread 142 diameter d8. Screw head 194 diameter d11 can be greater than screw thread 192 diameter d10. As also illustrated by FIG. 5, plate hole 118 diameter d3 can accommodate drill bit 142 with diameter d5, tap threads 182 with outer diameter d8, and screw threads 192 with outer diameter d10. Drill bit 142 can therefore translate through instrument hole 158 and plate hole 118 to drill attachment hole 108 to diameter d1′ corresponding to the inner thread diameter of screw threads 192. Tap threads 182 can translate through instrument hole 158 and plate hole 118 to tap attachment hole 108 to outer diameter d1 of screw threads 192. Screw 190 can translate through instrument hole 158 to cranial plate 112 where screw threads 192 may continue to translate through plate hole 118 and into attachment hole 108 thereby securing cranial plate 112 against occipital bone 104.

Screws 190, in one embodiment, can be driven into attachment holes one at a time in such a manner as to guide cranial plate 112 into abutting relationship with occipital bone 104. Starting with the centrally located attachment hole 108 a screw 190 can be partially driven into central attachment hole 108. Another screw 190 can be partially driven into one of the attachment holes distributed about central attachment hole 108. Then, in the current embodiment, a screw 190 can be partially driven into the attachment hole 108 opposite the other distributed attachment hole. Screws 190 may then be driven partially into the other distributed attachment holes 108. Following a similar order as in the foregoing steps, screws 190 can be driven the remainder of the way into attachment holes 108. Thus, as various screws 190 seat in attachment holes 108, cranial plate 112 can settle into the position for it desired by surgical personnel.

With continuing reference to FIG. 5, instrument hole 158, drill bit shank 144, tap shank 184 and screw head 194 can be dimensioned to cooperate to allow accurately locating, drilling, and tapping attachment hole 108 (of FIG. 1) and setting of screw 190 in attachment hole 108. Drill bit shank 144, tap shank 184, and screw head 194 can have, respectively, commonly sized diameters d6, d9, and d11 corresponding to instrument hole 158 diameter d7. Common diameters d6, d9, and d11 and corresponding diameter d7 can be such that when drill bit shank 144, tap shank 184, or screw head 194 are in instrument hole 158, walls of instrument hole 158 can maintain the particular device which is in instrument hole 158 in a pre-determined relationship with adapter 152, cranial plate 112, plate holes 106, and (more particularly) attachment holes 108. Attachment holes 108 created via use of instrument 150 (and adapter 152) can therefore correspond sufficiently accurately to hole pattern 116 of cranial plate 112 such that when screws 190 attach cranial plate 112 to occipital bone 104 stresses imparted to occipital bone 104 by screws 190 can be as low as practicable. Patients may therefore experience less discomfort, recovery time, etc. and the strength, functionality, and mechanical integrity of spinal stabilization system components that might be attached to cranial plate 112 can be maintained.

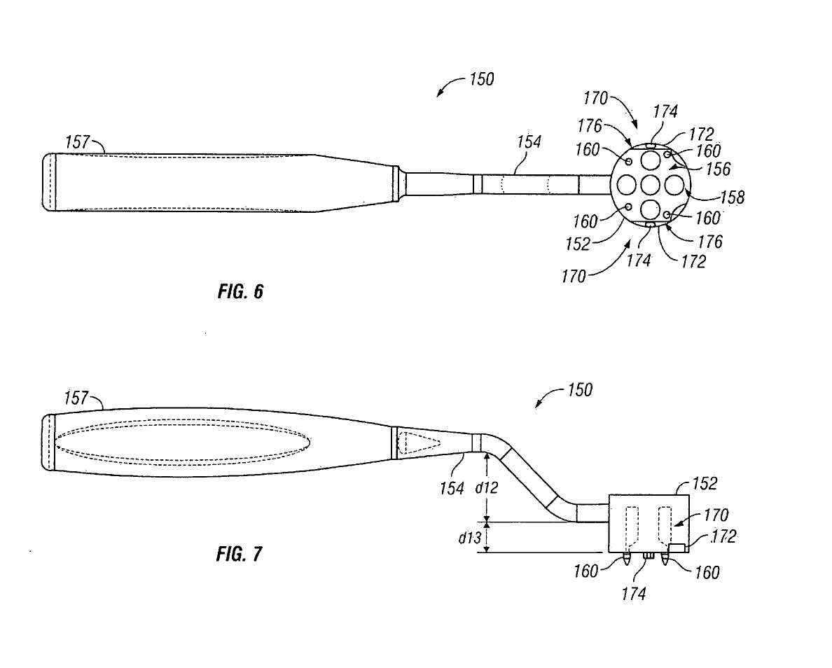

With reference now to FIG. 6, FIG. 6 illustrates a bottom plan view of instrument 150. Instrument 150 can include elongated handle 157, extension 154, and adapter 152. Handle 157 and extension 154 can allow surgical personnel to navigate adapter 152 (with or without cranial plate 112 attached there to) to positions adjacent to occipital bone 104. When cranial plate 112 is attached to instrument 150, surgical personnel can place cranial plate 112 at a selected location on occipital bone 104, drill and tap attachment holes 108, and drive screws 190 into attachment holes 108 to secure cranial plate 112 in place on occipital bone 104. Handle 157 can include knurls, grooves, ridges, grips, and other ergonomic features to aid surgical personnel in performing various operations associated with attaching cranial plates 112 to occipital bones 104 using instrument 150. FIG. 6 shows adapter 152 including hole pattern 156, instrument holes 158, alignment posts 160, attachment mechanism 170, resilient fingers 172, attachment posts 174, and slots 176 among other features of adapter 152.

With reference now to FIG. 7, FIG. 7 illustrates a side elevation view of one embodiment of instrument 150. As illustrated, instrument 150 can include handle 157, extension 154, and adapter 152. Extension 154 can offset handle 157 from adapter 152 by distance d12 in a direction perpendicular to handle 157. The position at which extension 154 couples to adapter 152 can further offset handle 157 from distal face 156 of adapter 152 by a distance d13 perpendicular to handle 157. Offset distances d12 (between handle 157 and adapter 152) and d13 (between distal face 156 and extension 154) can allow surgical personnel to place adapter 152 (and cranial plate 112) on occipital bone 104 while avoiding interference from anatomical features of the patient (such as shoulders) and surgical instruments and other surgical devices in the vicinity of occipital bone 104 and cervical spine 105. Offset distances d12 and d13 can also allow more convenient for surgical personnel to access instrument holes 118 for drilling and tapping attachment holes 108 and driving screws 190 into attachment holes 108. Offset distances d12 and d13 can also enable more convenient viewing and inspection of surgical sites associated with instrument 150. Instrument 150, as illustrated in FIG. 7, can include alignment posts 160 and attachment mechanisms 170 including resilient fingers 172, posts 174, gap 176, etc.

With reference now to FIG. 8, FIG. 8 illustrates a side elevation view of one embodiment of adapter 152 of instrument 150. As illustrated, adapter 152 can couple with extension 154, and can include posts 160 with one or more arcuate portions 162, and attachment mechanisms 170 including resilient fingers 172, attachment posts 174, and shoulders 177. Slots 176 between resilient fingers 172 and the body of adapter 152 are also illustrated by FIG. 8. Slots 176 can allow movement in generally lateral-medial directions of resilient fingers 172 to attach and release cranial plate 112 while providing access to surfaces 181 and 183 of resilient arms 172 and adapter 152 for cleaning, sterilization, maintenance, inspection, etc. Notches 178, which can correspond in shape and dimensions to generally medial portions of cranial plate 112 boss 117 can provide clearance for bosses 117 for attachment of cranial plate 112 to adapter 152 and detachment there from as illustrated by FIG. 8.

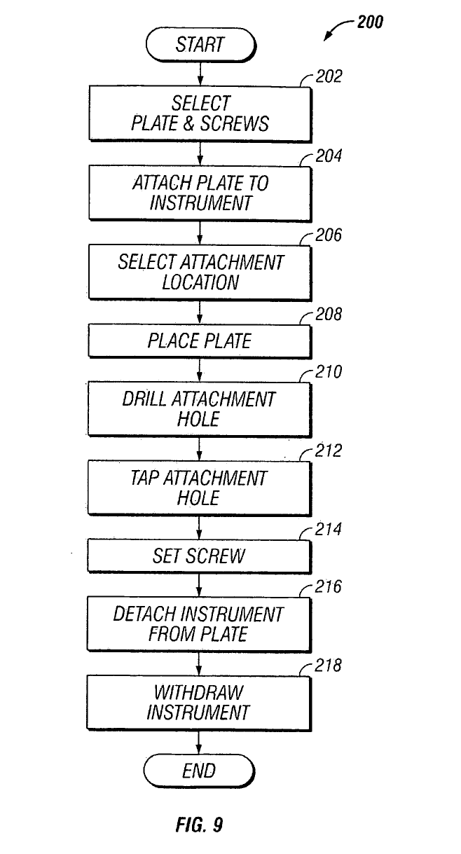

Now with reference to FIG. 9, FIG. 9 illustrates one embodiment of method 200 for attaching cranial plates 112 to occipital bones 104. At some time associated with method 200, surgical personnel can place the patient face down on a suitable operating surface, anesthetize and otherwise prepare the patient for surgery, and make an incision near occipital bone 104 when a posterior approach is to be used to attach cranial plate 112 to occipital bone 104. Surgical personnel may distract soft tissues (such s skin and muscles) from the surgical site, thereby allowing more convenient access to occipital bone 104 and surrounding anatomical structures. A surgical kit available to surgical personnel can include cranial plates 112 of various configurations and dimensions pre-determined to correspond to most, if not all, patient occipital bones 104. The surgical kit can include screws 190, bone anchors, etc. of various configurations and sizes pre-determined to yield potentially beneficial results when used in conjunction with one and other.

For instance, screws 190 of various lengths, head types, and thread designations can be included in the surgical kit. Cranial plates 112 and screws 190 can be made of various biocompatible materials such as titanium, stainless steel, zirconium, polymethyl methacryalate, etc Some screws 190 and cranial plates 112 can have plate hole 118 diameters d3 and screw thread 192 diameters d10, respectively, corresponding to each other. At step 202, surgical personnel can select cranial plate 112 and screws 190 (see FIG. 5) for attaching cranial plate 112 to occipital bone. 104. Selection of cranial plate 112 and screws 190 can be based on examination of the patient and more particularly occipital bone 104 and cervical vertebrae 105. Screws 190 can have common screw thread diameters d10 and common screw head 194 diameters d11. Cranial plate 112 can have plate holes 118 with diameters d3 corresponding to screw thread diameters d10. With continuing reference to FIG. 9, at step 204, surgical personnel can align selected cranial plate 112 with alignment posts 160 and attachment posts 174 on adapter 152 of instrument 150. Surgical personnel can urge cranial plate 112 toward distal face 156 of adapter 152, thereby causing resilient fingers 172 (see FIG. 4) to elastically yield allowing cranial plate 112 to contact distal face 156 thereby allowing resilient fingers to grasp cranial plate 112.

With cranial plate 112 attached to instrument 150, surgical personnel can use handle 157 of instrument 150 to navigate cranial plate 112 to a position generally adjacent to occipital bone 104 at step 206. Surgical personnel can evaluate occipital bone 104 and cranial plate 112 (in relatively close proximity to each other) and select a location on occipital bone 104 where their judgment indicates that the particular cranial plate 112 can be beneficially attached to occipital bone 104. Surgical personnel can place cranial plate 112 at accordingly using instrument 150 at step 208. With cranial plate 112 placed on occipital bone 104 and attached to instrument 150, pointed tips of alignment posts 160 (which can extend passed cranial plate 112), can grip the surface of occipital bone 104 and resist changes in position of cranial plate 112 during attachment of cranial plate 112 to occipital bone 104. Surgical personnel can inspect and evaluate cranial plate 112 and occipital bone 104 to determine whether the particular cranial plate 112 matches the contour, dimensions, and other features of occipital bone 104. When the particular cranial plate 112 does not match the contour, dimensions, and other features of occipital bone 104, surgical personnel can withdraw cranial plate 112 from the surgical site using instrument 150 and repeat the procedure with other cranial plates 112 until a suitable cranial plate 112 is found. When cranial plate 112 matches the contour, dimensions, and features of occipital bone 104 as determined by surgical personnel, surgical personnel can continue method 200.

At step 210, as illustrated by FIG. 9, surgical personnel can select drill bit 142 with diameter d5 corresponding to diameter d1′ of screw threads 192, diameter d8 of tap threads 182, and diameter d3 of plate hole 118 (see FIG. 5). Surgical personnel can attach drill bit 142 to extension 146 (which can have an internal driver for drill bit 142). Drill bit 142 can be generally aligned with instrument hole 158 and inserted into instrument hole 158 by surgical personnel. As drill bit 142 translates into and through instrument hole 158, drill bit shank 144 can encounter the walls of instrument holes 158. Because diameter d6 of drill bit shank 144 can correspond to diameterd 7 of instrument hole 158, instrument hole 158 can cause drill bit 142 to accurately align with plate hole 118. When desired, surgical personnel can continue advancing drill bit 142 into and through plate hole 118 and begin drilling attachment hole 108 into occipital bone 104.

When attachment hole 108 reaches a desired depth (for example, deep enough, to securely attach cranial plate 112 to occipital bone 104 yet shallow enough to not weaken occipital bone 104 or penetrate occipital bone 104 and underlying soft tissues), surgical personnel can withdraw drill bit 104 from occipital bone 104, cranial plate 112, and adapter 152 of instrument 150. In some embodiments, adapter 152 can include a hard stop positioned to interfere with drill bit shank 144 as drill bit shank 144 is advanced toward cranial plate 112. Thus, adapter 152 can stop drill bit shank 144 at a location set apart from cranial plate 112 thereby preventing drill bit shank 144 from contacting and marring cranial plate 112. Adapter 152 can also stop drill bit shank at a location corresponding to a desired depth of attachment hole 118; As desired, additional attachment holes 108 can be drilled at step 210 using other instrument holes 158 and plate holes 158 and 118. When desired, surgical personnel can inspect attachment holes 108 by peering through instrument hole 158 or by using an endoscope or other suitable viewing aid.

With instrument 150 and cranial plate 112 remaining in place, surgical personnel can generally align tap 180 with instrument hole 158 and advance it into instrument hole 158 at step 212. Diameter d9 of tap shank 184 (which can correspond to diameter d7 of instrument hole 158) can allow instrument hole 158 to accurately align tap threads 182 with plate hole 118 and attachment hole 108 in occipital bone 104. Attachment hole 108 can be threaded with tap 180. Surgical personnel can withdraw tap 180 from occipital bone 104, cranial plate 112, and adapter 152 of instrument 150 and, if desired, tap other attachment holes 108 in occipital bone 104. When desired, surgical personnel can inspect attachment holes 108 by peering through instrument hole 158 or by using an endoscope or other suitable viewing aid.

At step 214, surgical personnel can align screw 190 with instrument hole 158 manually or using a screwdriver which can retain screw 190 on the tip thereof. Surgical personnel can advance screw 190 into instrument hole 158 when desired. As screw threads 192 translate through instrument hole 158, diameter d11 of screw head 194 (which can correspond to diameter d7 of instrument hole 1.58), can allow the walls of instrument hole 158 to accurately align screw threads 192 with plate hole 118 and attachment hole 108 in occipital bone 104. Surgical personnel can continue advancing screw threads 190 through plate hole 118 and drive it into place in attachment hole 108 using a screwdriver or other instrument. When screw 190 has been driven as deeply as desired into attachment hole 104, surgical personnel can withdraw the screwdriver and inspect screw 190 by peering into instrument hole 158 or using an endoscope or other suitable viewing aids. When desired, surgical personnel can drive other screws 190 into other attachment holes 108.

When as many screws 190 have been driven into attachment holes 108 as desired, surgical personnel can detach instrument 150 from cranial plate 112 at step 216. To detach instrument 150 from cranial plate 112, surgical personnel can rotate handle 157 of instrument 150 to pivot instrument 150 partially about bosses 117 (in direction 159 illustrated by FIG. 3) of cranial plate 112 to overcome the grasping force being applied to cranial plate 112 by resilient fingers 172. Resilient fingers 172 can return to their relaxed positions generally adjacent adapter body 114 as instrument 150 withdraws from engagement with cranial plate 112.

Instrument 150 can be withdrawn from cranial plate 112 and occipital bone 104 at step 218. With instrument 150 withdrawn from occipital bone 104, surgical personnel can inspect cranial plate 112, screws 190, occipital bone 104, and cervical vertebrae C1-C7. Adjustments to occipital bone 104, cervical vertebrae C1-C7, cranial plate 112 and screws 190 can be made as surgical personnel might desire. If desired, instrument 150 may be used to navigate adapter 152 back to cranial plate 112 where it may be re-attached to cranial plate 112. With instrument 150 attached to cranial plate 112, screws 190 may be tightened, loosened, or removed from occipital bone 104 via instrument holes 158. If desired, cranial plate 112 can be removed also. When surgical personnel no, longer desire to use instrument 150, surgical personnel can clean and sterilize instrument 150 including surfaces 181 and 183 in slot 176 (see FIG. 8). When satisfied with attachment of cranial plate 112 to occipital bone 104, surgical personnel can attach stabilization rods and other stabilization devices to cranial plate 112 by bosses 117 and apertures 120. Surgical personnel can evaluate the stabilization rods and make can make adjustments to the, stabilization devices before closing the surgical site.

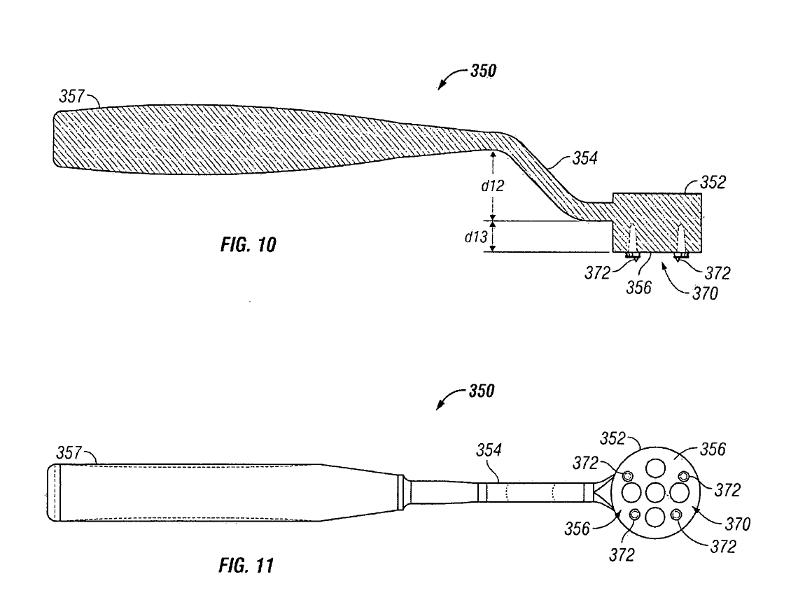

Attachment mechanisms 170 other than resilient fingers 172 and attachment posts 174 can be included in instrument 150 without departing from the scope of the disclosure. For instance, FIG. 10 depicts a side elevation view of one embodiment of instrument 350 including attachment mechanism 370 for attaching cranial plates 112 to occipital bones 104 (of FIG. 1). FIG. 10 shows that instrument 350 can include adapter 352, extension 354 (defining offset distances d12 and d13), and handle 357. Attachment mechanism 370 can include set screws 372 extending from distal face 356 of adapter 352. Split screws 372 can align cranial plate 112 and adapter 352 and, more particularly, hole pattern 116 of cranial plate 112 and a corresponding hole pattern of adapter 352. Split screws 372 can capture features of cranial plates 112 such as features 124 (see FIG. 4) to releasably attach cranial plate 112 to instrument 350. Split screws 372 can be removed from adapter 352 for cleaning and sterilization of split screws 372 and corresponding screw holes during cleaning and sterilization of instrument 350. FIG. 11 depicts a bottom plan view of instrument 350 including adapter 352, extension 354, distal face 356, handle 357, and attachment mechanism 370 including set screws 372.

Embodiments provide advantages over previously available approaches to attaching cranial plates. Cranial plates can be attached accurately and precisely using instruments of embodiments. Surgery can be efficiently conducted while errors caused by misaligned drill bits, taps, screws, etc can be eliminated or, at least, reduced by embodiments. Relative to each other, screws can be set more accurately and precisely by embodiments. Crowding of the surgical site with various instruments, drills, taps, portions of adjacent patient anatomy (such as the shoulders), etc. can be reduced by embodiments. In addition, the number of surgical steps and the number of surgical personnel involved in attaching cranial plates to occipital bones can be reduced by embodiments.

In the foregoing specification, specific embodiments have been described with reference to the accompanying drawings. However, as one skilled in the art can appreciate, embodiments of the anisotropic spinal stabilization rod disclosed herein can be modified or otherwise implemented in many ways without departing from the spirit and scope of the disclosure. Accordingly, this description is to be construed as illustrative only and is for the purpose of teaching those skilled in the art the manner of making and using embodiments of an anisotropic spinal stabilization rod. It is to be understood that the embodiments shown and described herein are to be taken as exemplary. Equivalent elements or materials may be substituted for those illustrated and described herein. Moreover, certain features of the disclosure may be utilized independently of the use of other features, all as would be apparent to one skilled in the art after having the benefit of this description of the disclosure.

1. A method comprising:
releasably attaching a cranial plate to a distal face of an adapter of an instrument, the cranial plate defining a pattern of holes;
placing the cranial plate on a cranium of a patient using the instrument;
drilling an attachment hole in the cranium using a drill bit having a shank and by engaging one of a pattern of instrument holes defined by the adapter body and corresponding to the pattern of cranial plate holes, the instrument holes being on the distal face and through the adapter body;
tapping the attachment hole in the cranium using a tap having a shank and by engaging the instrument hole with the tap shank; and
placing the screw in the attachment hole by engaging the instrument hole with the screw head, wherein the drill bit shank, the tap shank, and the screw head having a common diameter, the instrument hole having a diameter corresponding to the common diameter.

2. The method of claim 1 further comprising selecting the cranial plate from a set of cranial plates.

3. The method of claim 1 further comprising selecting the drill bit from a set of drill bits wherein each drill bit has a shank with the common diameter.

4. The method of claim 1 further comprising selecting the tap from a set of taps wherein each tap has a shank having the common diameter.

5. The method of claim 1 further comprising selecting the screw from a set of screws wherein each screw has a head having the common diameter.

6. The method of claim 1 wherein the placing the cranial plate includes using an elongated handle of the instrument coupled to the adapter and having an offset.

7. The method of claim 1 further comprising releasing the cranial plate from the instrument.

8. The method of claim 1 wherein the releasing the cranial plate further comprises releasing the cranial plate after the drilling and tapping of the attachment hole and the placing of the screw.

9. A kit comprising:
a set of cranial plates, each plate defining a pattern of holes;
a set of screws, each screw including a head;
a set of drill bits, each drill bit including a shank;
a set of taps, each tap including a shank, wherein the screw heads, the drill bit shanks, and the tap bodies have a common diameter; and
an instrument for use with the cranial plates and including:
an elongated handle; and
an adaptor coupled to the distal end of the handle and including a body and a distal face, the body defining a pattern of instrument holes on the distal face and corresponding to the pattern of cranial plate holes, the instrument holes being through the body and having a diameter corresponding to the common diameter.

10. The kit of claim 9 further comprising a plurality of retaining members on the instrument adjacent to the distal face and configured to releasably attach the cranial plate to the instrument.

11. The kit of claim 10 wherein the retaining members are resilient fingers.

12. The kit of claim 10 wherein the retaining members are split screws.

13. The kit of claim 9 further comprising a key on each of the distal faces and corresponding to a mating key on each of the cranial plates.

14. The kit of claim 9 wherein the instrument handle is offset from the center of the body.

15. An instrument for use with a cranial plate, a screw including a head, a drill bit including a shank, and a tap including a shank, the cranial plate defining a pattern of holes, the screw head, the drill bit shank, and the tap shank having a common diameter, the instrument comprising:
an elongated handle; and
an adaptor coupled to the distal end of the handle and including a body and a distal face, the body defining a pattern of instrument holes on the distal face and corresponding to the pattern of cranial plate holes, the instrument holes being through the body and having a diameter corresponding to the common diameter of the screw head, the drill bit shank, and the tap shank.

16. The instrument of claim 15 further comprising a plurality of retaining members adjacent to the distal face and being configured to releasably attach the cranial plate to the instrument.

17. The instrument of claim 15 wherein the retaining members are resilient fingers.

18. The instrument of claim 15 wherein the retaining members are split screws.

19. The instrument of claim 15 further comprising a key on the distal face corresponding to a mating key on the cranial plate.

20. The instrument of claim 15 wherein the handle is offset from the center of the body.
